(19) United States
(12) Patent Application Publication
(10) Pub. No.: US 2023/0240578 A1
(43) Pub. Date: Aug. 3, 2023

(54) ADHESIVE PAD FOR RETAINING AN ELECTRODE AGAINST A PATIENT'S SKIN

(71) Applicant: Seer Medical Pty Ltd, Melbourne, Victoria (AU)

(72) Inventors: Kyle Damon SLATER, Kensington, Victoria (AU); Rory KNIGHT-SADLER, Mount Evelyn, Victoria (AU); Dean Robert FREESTONE, West Melbourne, Victoria (AU); Kristan MARLOW, Abbotsford, Victoria (AU)

(73) Assignee: Seer Medical Pty Ltd, Melbourne, Victoria (AU)

(21) Appl. No.: 18/004,711

(22) PCT Filed: Jul. 16, 2021
(86) PCT No.: PCT/AU2021/050767

§ 371 Date: Jan. 9, 2023

(30) Foreign Application Priority Data
Jul. 17, 2020 (AU) 2020902484

(51) Int. Cl. A61B 5/257; A61B 5/00

(52) U.S. Cl. CPC A61B 5/257; A61B 5/6833; A61B 2562/164; A61B 5/291

(57) ABSTRACT

Adhesive pad (10) for retaining an electrode against a patient's skin. The pad (10) includes an adhesive gel layer (16) for adhering to the skin, and a non-adhesive layer (18) carrying the gel layer (16). The gel layer (16) is configured to be water-soluble and define a non-tacky external shell. Methods for producing an adhesive pad for adhering to a patients skin, and tools for pressing an adhesive pad to a patients skin, are also disclosed.

### TECHNICAL FIELD

The present disclosure relates, generally, to skin-friendly adhesive pads and, particularly, to adhesive pads for adhering to a patient's skin to allow retaining an electrode against the skin for a prolonged period.

### BACKGROUND

Patients are often monitored using an electrode array coupled to skin, for example, to obtain electroencephalogram (EEG) or electrocardiogram (ECG) data. Monitoring brain activity using EEG recording is performed by coupling the array of electrodes to a patient's scalp. Monitoring cardiac activity using ECG recording is performed by coupling the array of electrodes to the patient's chest. The electrodes are secured by either a dry connection, typically being carried by a cap or adhesive foam pads, or an adhesive applied directly to each electrode. Data is recorded using the electrodes whilst the patient is static, referred to as bedside monitoring, or whilst moving, referred to as ambulatory monitoring. The duration of ambulatory monitoring of the patient often requires between four and ten days.

To reliably retain the electrode array against a patient's skin for a prolonged period, such as during ambulatory monitoring, an adhesive, typically being collodion glue, is used to adhere each electrode to the skin. Whilst this provides a secure connection between the electrode and the patient, commonly used adhesives, such as collodion glue, often cause skin irritation. To remove the electrode array at the end of the monitoring period, a chemical solution, such as acetone, is typically applied to each electrode to dissolve the glue. This often exacerbates skin irritation, causing discomfort and/or pain. Furthermore, commonly used material, such as collodion glue and acetone, are noxious.

Any discussion of documents, acts, materials, devices, articles or the like which has been included in the present specification is not to be taken as an admission that any or all of these matters were common general knowledge in the field relevant to the present disclosure as it existed before the priority date of each of the appended claims.

### SUMMARY

According to one aspect of the disclosure, there is provided an adhesive pad for retaining an electrode against a patient's skin. The pad includes an adhesive gel layer for adhering to the skin, and a non-adhesive layer carrying the gel layer. The gel layer is configured to be water-soluble and define a non-tacky external shell.

The gel layer may comprise dehydrated water-soluble adhesive gel.

The gel layer may be further configured such that, responsive to submerging the gel layer in water for a defined period, at least a portion of the gel layer is rehydrated to be malleable and tacky.

The gel layer may define an electrode portion dimensioned to cover and extend beyond the electrode to allow adhering the electrode portion to the electrode and the skin, and a tail portion extending from the electrode portion, the tail portion dimensioned to cover and extend beyond a portion of a wire attached to the electrode to allow adhering the tail portion to the wire and the skin.

The gel layer may define a peripheral profile surrounding the electrode portion and the tail portion, such that the peripheral profile has a teardrop shape.

The non-adhesive gel layer may be configured as a flexible substrate.

According to another aspect of the disclosure, there is provided a method for producing an adhesive pad for adhering to a patient's skin. The method includes: forming viscous water-soluble adhesive gel to define a gel layer having a specific shape; joining a non-adhesive substrate to the gel layer; and dehydrating the gel layer to define a non-tacky external shell, such that the gel layer and non-adhesive substrate form the adhesive pad. It will be appreciated that the steps of the method may be executed in a different order to the above, and two or more steps may be executed simultaneously.

Forming the gel may include introducing the gel into a cavity defined by a mould, where the cavity defines the specific shape.

Joining the substrate to the gel layer may include placing the substrate within the cavity before introducing the gel into the cavity, and introducing the gel into the cavity may include arranging the gel to at least partly cover the substrate.

Dehydrating the gel layer may include a first dehydration stage where the gel layer is dehydrated whilst arranged in the cavity, an intermediary stage where the gel layer and the substrate are removed from the cavity, and a second dehydration stage where the gel layer is dehydrated when removed from the cavity.

A duration of the first dehydration stage may be substantially greater than a duration of the second dehydration stage.

According to a further aspect of the disclosure, there is provided a tool for pressing an adhesive pad to a patient's skin. The tool includes a body defining a central axis and a handle extending from the body along the central axis. The body defines a recess symmetrically arranged about the axis and shaped to receive the adhesive pad. The body is at least partly formed from a transparent material so that, in use, the adhesive pad is visible when arranged within the recess.

The body and the handle may be formed from the transparent material.

The body may define a rim around a periphery of the recess, the rim defining a notional plane, and the body may define one or more external sidewalls at least partially surrounding the rim and extending perpendicular, or at an acute angle, to the notional plane.

The handle may have opposed ends, each end being connected to the body to form a loop extending along the central axis.

The handle may be arranged to define a void between the handle and the body, the void dimensioned to allow a user to insert a finger therethrough to grip the handle.

The body may define an aperture opening out into the recess, the aperture dimensioned to allow at least partially receiving the electrode.

The body may define a pair of parallel ribs extending from the recess, the ribs spaced apart to allow at least partially receiving a portion of a wire attached to the electrode.

The body may define a slot opening out into the recess, the slot dimensioned to at least partially receive the portion of the wire, and the ribs are spaced either side of the slot and shaped to diverge away from the slot.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

It will be appreciated embodiments may comprise steps, features and/or integers disclosed herein or indicated in the specification of this application individually or collectively, and any and all combinations of two or more of said steps or features.

### BRIEF DESCRIPTION OF DRAWINGS

Embodiments will now be described by way of example only with reference to the accompany drawings in which:

FIGS. 1 and 2 are perspective and cross-section views, respectively, of an adhesive pad for retaining an electrode against a patient's skin;

FIG. 3 is a flow chart illustrating process steps to manufacture the adhesive pad shown FIGS. 1 and 2;

FIGS. 4 and 5 are perspective and plan views, respectively, of a quantity of the adhesive pads sufficient to allow fitting an EEG electrode array to a single patient;

FIG. 6 is a flow chart illustrating process steps to apply the adhesive pads shown in FIG. 4 to a patient;

FIGS. 7 and 8 are perspective and plan views, respectively, of a tool for pressing the adhesive pad to the patient's skin; and

FIG. 9 is a cross-section view taken along line A-A in FIG. 8.

### DESCRIPTION OF EMBODIMENTS

In the drawings, reference numeral 10 generally designates an adhesive pad 10 for retaining an electrode against a patient's skin. The pad 10 includes an adhesive gel layer 16 for adhering to the skin, and a non-adhesive layer 18 carrying the gel layer 16. The gel layer 16 is configured to be water-soluble and define a non-tacky external shell.

It will be appreciated that the non-tacky configuration of the external shell of the gel layer 16 means that the shell is substantially, or entirely, not sticky to touch. In other words, the shell is substantially non-adhesive, such that external surfaces of the layer 16 do not adhere to other objects. It will also be appreciated the shell may be configured to completely, or only partially, enclose the gel layer 16 carried on the non-adhesive layer 18.

The adhesive pad 10 is described below with reference to using the pad 10 to secure an electrode to a patient's scalp to allow ambulatory EEG monitoring over a prolonged period, such as a week. It will be appreciated that the adhesive pad 10 is suitable for other applications requiring adhesion to skin for a prolonged period, such as securing an electrode or other circuitry to a patient's chest to allow ambulatory ECG monitoring, or for mounting to other skin-covered body parts. Similarly, the adhesive pad 10 is configurable to secure or encapsulate discrete electronic devices to the patient's skin, such as a motion sensor, to allow alternative monitoring. In such embodiments, the sensor is typically configured as a disposable, stand-alone device which communicates data wirelessly, and is embedded in the pad 10.

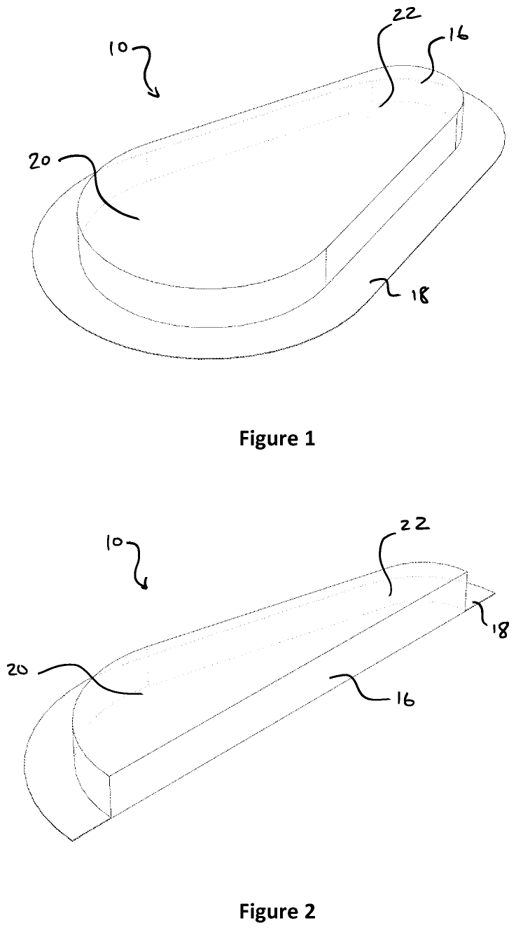

FIGS. 1 and 2 show the adhesive pad 10 and illustrate the arrangement of the layers 16, 18. Each layer 16, 18 is configured to be planar and define a peripheral profile configured so that the periphery of the non-adhesive layer 18 is offset from the periphery of the gel layer 16 to define a larger surface area than the gel layer 16. In other embodiments (not illustrated), the peripheral profile of the gel layer 16 is dimensioned to extend beyond or be coincident with the peripheral profile of the non-adhesive layer 18.

The adhesive gel layer 16 defines an electrode portion 20, dimensioned so that, in use, the portion 20 covers and extends beyond the electrode to allow adhering to the patient's skin. The gel layer 16 also defines a tail portion 22 extending from the electrode portion 20 and dimensioned so that, in use, the portion 22 covers and extends beyond part of a wire attached to the electrode. The portions 20, 22 are bound within the peripheral profile of the layer 16 which defines a teardrop shape.

In the illustrated embodiment, the adhesive layer 16 is formed from dehydrated water-soluble adhesive gel, as described in greater detail below. The non-adhesive layer 18 is formed from a flexible, and typically non-stick, substrate, such as silicone-coated paper or polymeric sheet. In some embodiments (not illustrated), the non-adhesive layer 18 comprises a polymeric cup or frame to allow defining structure in the layer 18. In other embodiments (not illustrated), the non-adhesive layer 18 is formed from the same material as the adhesive layer 16 and arranged to avoid rehydration prior to application of the pad 10 to the patient, as described below, such that the later 18 remains non-adhesive. In yet other embodiments (not illustrated), the non-adhesive layer comprises non-adhesive granular material, such as sand or polymeric pellets, embedded in a face of the adhesive layer 16 such that the face is substantially non-adhesive.

Manufacture of the adhesive pad 10 generally involves the following steps, but not necessarily in this order, or executed sequentially: forming viscous water-soluble adhesive gel to define the gel layer 16 having a specific shape; joining the non-adhesive substrate 18 to the gel layer 16; and dehydrating the gel layer 16 to define a non-tacky external shell.

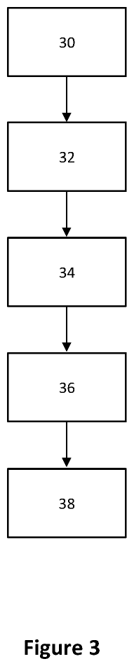

FIG. 3 is a flow chart illustrating process steps to manufacture the adhesive pad 10. Initially, at stage 30, a mould (not illustrated) defining a cavity which defines the specific shape is provided, and the non-adhesive substrate 18 is arranged within the cavity. Typically, the mould is configured to define a plurality of cavities to allow forming many pads 10 simultaneously. Each cavity is sprayed with a release agent, such as canola oil. The substrate 18 is defined by a profile scored or perforated, such as with a laser cutter, into a sheet of substrate material, which defines a complementary plurality of substrates 18 to the cavities, and the sheet is sandwiched between an upper and lower portion of the mould such that each substrate 18 is secured at a base of one of the cavities.

At stage 32, the water-soluble adhesive gel is introduced into the cavity. At this stage, the gel is a viscous liquid. Introducing the gel into the cavity involves pouring, spraying and/or injecting the gel, depending on the viscosity of the gel. A sufficient quantity of the gel is introduced to each cavity to cover the substrate 18.

At stage 34, the mould is moved into a chamber of a dehydrator (not illustrated). The chamber is heated between 30° C. and 70° C., and typically to 50° C., and operated for 2-6 hours, and typically for 4 hours. This causes moisture to be extracted from the gel layer 16, and the gel layer 16 to adhere to the substrate 18. The mould is then removed from the dehydrator and allowed to cool.

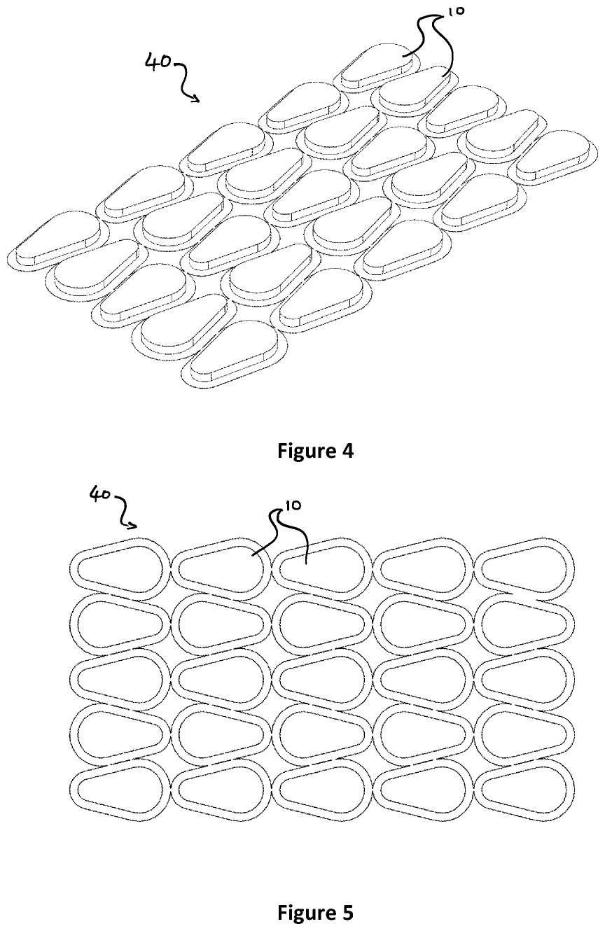

At stage 36, the pads 10, each comprising one of the substrates 18 joined to and carrying a gel layer 16, are removed from the mould. The scoring profile of the sheet is configured such that a set 40 (FIGS. 4 and 5) of twenty-five substrates 18 and associated gel layers 16 remain connected when removed from the mould. The sheet is configured in this way to ensure the pads 10 are grouped in sets 40 of twenty-five pads 10 which are sufficient to allow securing a conventional EEG electrode array, comprising twenty-five electrodes, to a single patient. The pads 10 are then returned to the chamber of the dehydrator which is operated, for example, by heating to around 55° C. for 30 minutes. This causes further moisture to be extracted from the gel layer 16 allowing dehydration of parts of the layer 16 which were previously in contact with the mould.

At stage 38, the pads 10 are removed from the dehydrator and allowed to cool. The gel layer 16 of each pad 10 is now at least partially dehydrated to define the non-tacky external shell. Depending on the configuration of the dehydration stages 34, 36, the gel layer 16 may have a viscous liquid gel core surrounded by the non-tacky shell.

The non-tacky shell allows handling and packaging of each set 40 of twenty-five pads 10 without the adhesive layers 16 adhering to the handler or the packaging. Each set 40 is inserted into a bag (not illustrated) which is then sealed. The sealed bag is packaged in a box (not illustrated) and labelled. In the packaged state, each adhesive layer 16 defines a substantially resiliently deformable structure which allows flex. It will be appreciated that, depending on the configuration of the dehydration stages 34, 36, the gel layer 16 may be substantially dehydrated to define a substantially rigid structure.

It will be appreciated that the stages 30-38 described above are exemplary and that executing these stages in an alternative order, or executing alternative stages to produce the adhesive pad 10, is within the scope of this disclosure. For example, in some embodiments (not illustrated), stage 32, involving moulding the gel layer 16, is executed before stage 30, attaching the substrate 18 to the gel layer 16. In such embodiments, the gel is introduced into the cavities of the mould before the substrates 18 are placed on to the gel within the cavities, whilst the gel is viscous. The mould is then moved into the dehydrator at stage 34 to allow dehydrating the gel layers 16 and cause joining to the substrates 18.

In other embodiments (not illustrated), the mould is configured to be temperature controlled, such as by defining conduits containing fluid. In such embodiments, the initial dehydration stage 34 involves heating the mould, by conveying heated fluid through the conduits, in addition to operating a fan to cause air flow across or through the mould, to dehydrate the gel layers 16. In these embodiments, the second dehydration stage 36 may not be necessary depending on the configuration of the initial dehydration stage 34.

In some embodiments (not illustrated), the mould is configured to define one or more rectangular-shaped cavities to allow moulding a large sheet of the gel on to the sheet of substrates 18 at stage 32. The gel sheet is then dehydrated at stage 34 to join to the plurality of substrates 18 defined by the sheet. At stage 36, the gel sheet, adhered to the sheet of substrates 18, is removed from the cavity, and groups of twenty-five substrates 18 are cut or punched out of the gel sheet thereby forming the adhesive pads 10.

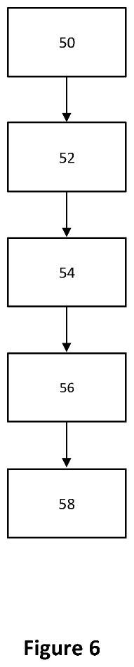

FIG. 6 is a flow chart illustrating process steps to apply the set 40 of adhesive pads 10 to a patient's scalp. Initially, at stage 50, the set 40 of pads 10 are removed from the bag, separated from each other, and at least some of the pads 10 are arranged on a dipping tray (not illustrated).

At stage 52, a water bath (not illustrated) is heated to 50° C. and the dipping tray is submerged in the water bath for three minutes. This causes an outer layer of the gel layer 16 to be heated and rehydrated, consequently transforming this outer layer to a malleable, tacky state. Also, due to the heat of the bath, the resilience of the gel layer 16 is reduced which increases flexibility of the pad 10.

At stage 54, the pads 10 are removed from the water bath and arranged in a humid environment to maintain the tacky outer surface of each gel layer 16. Typically, this involves securing the dipping tray above the water bath and covering the bath and try with a cover.

At stage 56, the patient's scalp is prepared, typically involving cleaning the skin to remove oil and dry skin. Conductive paste is then applied to an EEG electrode and the electrode is placed against the patient's scalp.

At stage 58, one of the pads 10 is placed over the electrode such that the electrode portion 20 of the gel layer 16 is covering the electrode and the tail portion 22 of the gel layer 16 is covering the wire attached to the electrode. The pad 10 is then pressed against the electrode and the scalp, typically for 10 seconds. Pressing the pad 10 causes the rehydrated portion of the adhesive layer 16 to deform and adhere to the scalp. To standardise the fitting process, which enhances accuracy and efficiency, pressing the pad 10 against the patient and electrode typically involves using the pressing tool 60, described in greater detail below.

Depending on the skill level of the clinician applying the pads 10, the entire set 40 may be rehydrated together and then applied to the scalp, or this may be done in batches of around five pads 10 requiring repetition of the rehydration steps 52, 54.

The gel layer 16 is then allowed to dry (cure) so that external facing surfaces, which are in contact with air, become non-tacky and the portion of the gel layer 16 in contact with the skin is firmly adhered to the scalp. During the drying period, the substrate 18 is arranged outwardly to inhibit other objects, such as hair, also adhering to the gel layer 16. As only a thin, peripheral region of the gel layer 16 is rehydrated at stage 52, the gel layer 16 cures rapidly, meaning that the drying period is short, typically being around 1-2 hours.

Once all of the electrodes 12 in the array are secured to the patient's scalp with the pads 10 at stage 58, EEG monitoring commences. During monitoring, a patient is required to prevent the pads 10 from becoming wet. This includes wearing a shower cap when washing. Also, during monitoring, additional conductive paste may be inserted into each pad 10 to replenish paste where impedance has become non-optimal, for example, by melting and escaping from under the electrode and pad 10, or by being fractured. This is achieved by operating a paste applicator (not illustrated) to pierce the pad 10 and insert the paste. Due to the resilience of the gel layer 10, the layer 10 re-seals the perforation caused by the applicator. When the monitoring period is complete, the patient removes the electrode array and pads 10 by showering, or otherwise wetting the scalp.

It will be appreciated that the stages 50-58 described above are exemplary and that executing these stages in an alternative order, or executing alternative stages to apply the adhesive pad 10 to the patient's scalp, or other region of skin, is within the scope of this disclosure. For example, in some embodiments (not illustrated), the rehydration stage 52 involves placing the set of pads 10 in a chamber of a humidifier where the chamber contains heated water vapour. In such embodiments, the following stage 54 is not necessary as the pads 10 may be stored in the humidifier until application to the patient is required.

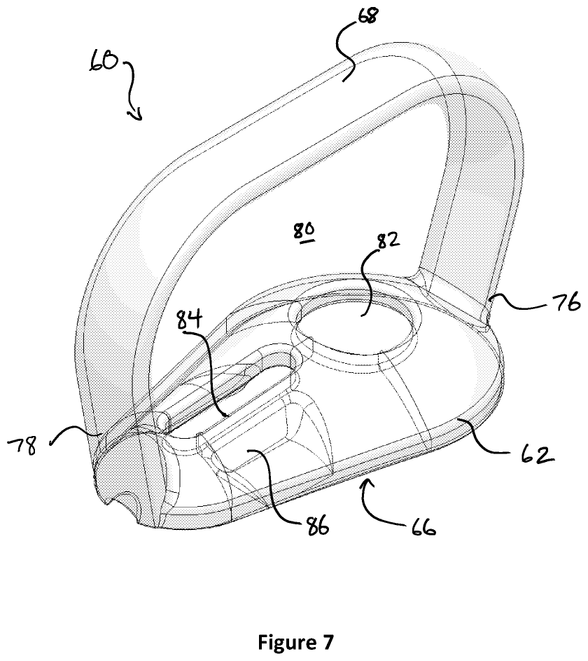

FIGS. 7 to 9 illustrate the pressing tool 60 which is configured to enhance application of one of the adhesive pads 10 to the patient's skin to secure an electrode in place. The tool 60 includes a body 62 defining a central axis 64 and a recess 66 symmetrically arranged about the axis 64 and shaped to receive the adhesive pad 10, and a handle 68 extending from the body 62 along the central axis 64. The body 62 is at least partly formed from a transparent material so that, in use, the adhesive pad 10 is visible when arranged within the recess 66.

In the illustrated embodiment, the entire body 62 and handle 68 are formed from transparent material, such as polypropylene. This usefully enhances visibility of the pad 10 under the tool 60 whilst applying the pad 10 to the patient's skin. In other embodiments (not illustrated) only part of the body 62 is configured to be transparent, typically being a peripheral region substantially surrounding the recess 66 to allow the user to align the recess 66 with the pad 10.

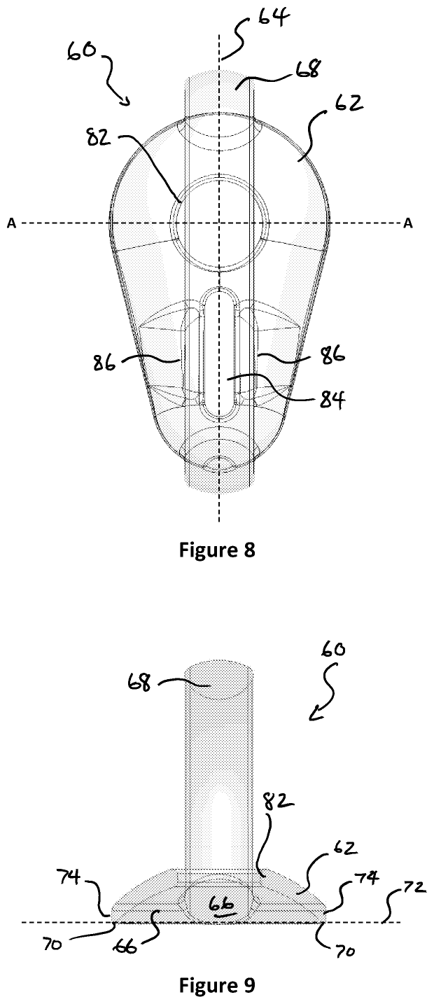

Best shown in FIG. 9, being a cross-section along line A-A of FIG. 8, the body 62 defines a rim 70 around a periphery of the recess 66. The rim 70 defines a notional plane 72. In the illustrated embodiment, the body 62 defines a sidewall 74 substantially surrounding the rim 70 and extending perpendicularly to the notional plane 72. In other embodiments (not shown), the body 62 defines more than one sidewall 74 to at least partially surround the rim 70, and each sidewall 74 is arranged at an acute angle relative to the notional plane 72 to incline away from the plane 72. Either arrangement of the sidewalls 74 relative to the plane 72 minimises a separation distance of the periphery of the body 62 and the periphery of the pad 10 when arranged within the recess 66. This usefully assists a user to align the peripheries when arranging the tool 60 over the pad 10 before urging the pad 10 against the skin, consequently enhancing application of the pad 10 to the skin.

Best shown in FIG. 7, the handle 68 defines opposed ends 76, 78, each being connected to the body 62 to form a loop along the axis 64. This arrangement defines a void 80 between the handle 68 and the body 62, the void 80 being dimensioned to receive a user's finger to allow gripping the handle. This allows the handle 68 to be gripped between a finger arranged through the void 80 and a thumb, which is most suitable for user's having small hands, or the handle 68 to be pinched on opposed sides between a finger and thumb, which is most suitable for user's having large hands. In either scenario, the arrangement of the handle 68 places the user's hand such that it does not obstruct viewing the periphery of the body 62, consequently enhancing alignment of the tool 60 with the pad 10.

The body 62 defines an aperture 82 and a slot 84. In the illustrated embodiment, the aperture 82 and the slot 84 extend through the body 62 to open out into the recess 66. The aperture 82 is dimensioned to at least partially receive the electrode, and the slot 84 is dimensioned to at least partially receive the wire attached to the electrode, when the tool 60 is urged against the electrode and the pad 10. In other embodiments (not illustrated), the aperture 82 and/or the slot 84 extend from the recess 66 partway through the body 62 to define further recesses.

The body 62 also defines a pair of ribs 86 extending from the recess 66 to be spaced either side of the slot 84. The ribs 86 are arranged to diverge away from the slot 84 to assist forming the gel layer 16 around the wire. This enhances forming a secure bond between the pad 10 and the wire.

The adhesive pad 10 is configured to transform between two states: a packaged state, where the gel layer 16 has a non-tacky shell, and an application state, where external surfaces of the gel layer 16 are malleable and tacky. This advantageously enhances handling and packaging of the pads 10, as the pads 10 do not stick to the handler or packaging, and enhances application of the pads 10 to the patient, as the gel layer 16 is easily deformed to conform to the profile of the skin, penetrates through hair to adhere to the skin, and dries rapidly. Furthermore, the non-adhesive layer 18 inhibits objects, such as hair, being adhered to the gel layer 16 before it has cured and dried.

The gel layer 16 of the pad 10 is configured to be water soluble. This allows a patient to dissolve the layer 16 by application of water. This is a convenient, pain-free method of removing the pad 10 and the associated electrode which can be performed in the comfort of the patient's home, reducing the burden on clinics or hospitals.

Manufacture of the adhesive pad 10 involves dehydrating the gel layer 16 such that it defines non-tacky external surfaces. Employing a dehydration stage usefully causes the external surfaces of the layer 16 to dry and form a non-tacky shell without causing the entire layer 16 to cure. This means that when the layer 16 is rehydrated and heated, the layer 16 becomes at least partially malleable and tacky, meaning that the layer 16 can be pressed into place and allowed to cure to adhere to the patient's skin.

The pressing tool 60 defines the recess 66 which is shaped to receive the adhesive pad 10, and is at least partially transparent to allow viewing the pad 10 when arranged in, or under, the recess 66. This arrangement enhances application of the pad 10 to the skin by ensuring appropriate force is applied across the pad 10 and/or assist with forming the adhesive layer 16 around the electrode and wire and against the skin. Furthermore, using the tool 60 standardises the application process meaning that users require minimal or no training to learn how to apply the pads 10 to secure an EEG electrode array to the scalp.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. An adhesive pad for retaining an electrode against a patient's skin, the pad including:
an adhesive gel layer for adhering to the skin, the gel layer configured to be water-soluble and define a non-tacky external shell; and
a non-adhesive layer carrying the gel layer.

2. The adhesive pad of claim 1, wherein the gel layer comprises dehydrated water-soluble adhesive gel.

3. The adhesive pad of claim 2, wherein the gel layer is further configured such that, responsive to submerging the gel layer in water for a defined period, at least a portion of the gel layer is rehydrated to be malleable and tacky.

4. The adhesive pad of claim 1, wherein the gel layer defines an electrode portion dimensioned to cover and extend beyond the electrode to allow adhering the electrode portion to the electrode and the skin, and a tail portion extending from the electrode portion, the tail portion dimensioned to cover and extend beyond a portion of a wire attached to the electrode to allow adhering the tail portion to the wire and the skin.

5. The adhesive pad of claim 4, wherein the gel layer defines a peripheral profile surrounding the electrode portion and the tail portion, the peripheral profile having a teardrop shape.

6. The adhesive pad of claim 1, wherein the non-adhesive gel layer is configured as a flexible substrate.

7. A method for producing an adhesive pad for adhering to a patient's skin, the method including:
forming viscous water-soluble adhesive gel to define a gel layer having a specific shape;
joining a non-adhesive substrate to the gel layer; and
dehydrating the gel layer to define a non-tacky external shell, such that the gel layer and non-adhesive substrate form the adhesive pad.

8. The method of claim 7, wherein forming the gel includes introducing the gel into a cavity defined by a mould, the cavity defining the specific shape.

9. The method of claim 8, wherein joining the substrate to the gel layer includes placing the substrate within the cavity before introducing the gel into the cavity, and wherein introducing the gel into the cavity includes arranging the gel to at least partly cover the substrate.

10. The method of claim 8, wherein dehydrating the gel layer includes a first dehydration stage where the gel layer is dehydrated whilst arranged in the cavity, an intermediary stage where the gel layer and the substrate are removed from the cavity, and a second dehydration stage where the gel layer is dehydrated when removed from the cavity.

11. The method of claim 10, wherein a duration of the first dehydration stage is substantially greater than a duration of the second dehydration stage.

12. A tool for pressing an adhesive pad to a patient's skin, the tool including:
a body defining a central axis and a recess symmetrically arranged about the axis and shaped to receive the adhesive pad, the body being at least partly formed from a transparent material so that, in use, the adhesive pad is visible when arranged within the recess; and
a handle extending from the body along the central axis.

13. The tool of claim 12, wherein the body and handle are formed from a transparent material.

14. The tool of claim 12 or 13, wherein the body defines a rim around a periphery of the recess, the rim defining a notional plane, and the body defines one or more external sidewalls at least partially surrounding the rim and extending perpendicular, or at an acute angle, to the notional plane.

15. The tool of claim 12, wherein the handle has opposed ends, each end being connected to the body to form a loop extending along the central axis.

16. The tool of claim 12, wherein the handle is arranged to define a void between the handle and the body, the void dimensioned to allow a user to insert a finger therethrough to grip the handle.

17. The tool of claim 12, wherein the body defines an aperture opening out into the recess, the aperture dimensioned to allow at least partially receiving an electrode.

18. The tool of claim 12, wherein the body defines a pair of parallel ribs extending from the recess, the ribs spaced apart to allow at least partially receiving a portion of a wire attached to an electrode.

19. The tool of claim 18, wherein the body defines a slot opening out into the recess, the slot dimensioned to at least partially receive the portion of the wire, and the ribs are spaced either side of the slot and shaped to diverge away from the slot.
